(19) United States
(12) Patent Application Publication
(10) Pub. No.: US 2021/0007814 A1
(43) Pub. Date: Jan. 14, 2021

(54) ENABLING THIRD-PARTY APPLICATION EXECUTION ON ROBOTIC SURGICAL SYSTEMS

(71) Applicant: Verily Life Sciences LLC, South San Francisco, CA (US)

(72) Inventors: James Shuma, San Jose, CA (US); Robert Tsai, San Francisco, CA (US)

(73) Assignee: Verily Life Sciences LLC, South San Francisco, CA (US)

(21) Appl. No.: 16/507,714

(22) Filed: Jul. 10, 2019

(51) Int. Cl. A61B 34/35; A61B 90/50; A61B 34/00; A61B 90/00

(52) U.S. Cl. CPC A61B 34/35; A61B 90/50; A61B 2017/00199; A61B 90/361; A61B 34/25; A61B 34/74

(57) ABSTRACT

Enabling third-party application execution on robotic surgical systems are disclosed. One disclosed example computing device includes a processor; and a non-transitory computer readable medium configured to store at least executable instructions, wherein the executable instructions, when executed by the processor, cause the computing device to: receive a request to execute an application from a robotic surgical system; obtain a cryptographic signature associated with a manifest, the manifest associated with the application; in response to verification of the manifest or the application based on the cryptographic signature, determine at least one configuration setting for the application based on the manifest, the at least one configuration setting comprising a permission; configure an execution environment based on the at least one configuration setting, the at least one configuration setting enabling the application to interact with the robotic surgical system; and execute the application in the execution environment.

### FIELD

The present application generally relates to robotic surgery and third-party applications, and more particularly relates to robotic surgery enabling third-party application execution in a restricted environment.

### BACKGROUND

The use of robotic tools to assist with surgical procedures is becoming increasingly common. The robotic tools may assist with any aspect of surgery, including but not limited to insertion or removal of surgical instruments, resection of tissue, insertion of implanted devices, etc. Such robotic tools may be used for a variety of reasons, including because their movements may be more precise and less prone to transient movements. During a robotic surgery, the surgeon may be presented with visual information to help guide the procedure, such as video from an endoscope.

### SUMMARY

Various examples are described for enabling third-party application execution on robotic surgical systems. One example computing device includes a processor; and a non-transitory computer readable medium configured to store at least executable instructions, wherein the executable instructions, when executed by the processor, cause the computing device to: receive a request to execute an application from a robotic surgical system; obtain a cryptographic signature associated with a manifest, the manifest associated with the application; in response to verification of the manifest or the application based on the cryptographic signature, determine at least one configuration setting for the application based on the manifest, the at least one configuration setting comprising a permission; configure an execution environment based on the at least one configuration setting, the at least one configuration setting enabling the application to interact with the robotic surgical system; and execute the application in the execution environment.

One example method includes receiving, by a computing device, a request to execute an application from a robotic surgical system; obtaining, by the computing device, a cryptographic signature associated with a manifest, the manifest associated with the application; determining, by the computing device and in response to verification of the manifest or the application based on the cryptographic signature, at least one configuration setting for the application based on the manifest, the at least one configuration setting comprising a permission; configuring, by the computing device, an execution environment based on the at least one configuration setting, the at least one configuration setting enabling the application to interact with the robotic surgical system; and executing the application in the execution environment.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 shows an example robotic surgical system enabling third-party application execution in a restricted environment.

FIG. 2 shows an example robotic surgical system enabling third-party application execution in a restricted environment.

FIG. 3 shows an example robotic surgical system enabling third-party application execution in a restricted environment.

FIG. 4 shows an example station of a robotic surgical system enabling third-party application execution in a restricted environment.

FIG. 5 shows an example method for a robotic surgical system enabling third-party application execution in a restricted environment.

FIG. 6 shows an example method for a robotic surgical system enabling third-party application execution in a restricted environment.

FIG. 7 shows an example computing device for a robotic surgical system enabling third-party application execution in a restricted environment.

### DETAILED DESCRIPTION

Examples are described herein in the context of robotic surgical systems enabling third-party application execution in a restricted environment. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

During surgery, it may be helpful to have certain types of functionality available to assist the surgeon or other medical personnel during the surgical procedure. For example, a surgeon may desire to consult with a specialist via a telepresence from a remote location. In some examples, a surgeon may be interested in accessing medical images, e.g., X-rays, CT scan images, MRI images, PET scans, etc. from one or more patients records. In other examples, it may be desirable to include videos of similar surgical procedures on other patients, medical reports, medical studies, or other medical research information.

However, obtaining such information during the surgery can be problematic. In particular, if the images or video are not already available on the robotic surgical system for viewing, the surgeon or other medical personnel in the operating room (“OR”) may need to identify image information of interest and instruct the robotic surgical system to retrieve and view those images, videos, or other imaging information. This may interrupt the surgical procedure, if the images are needed mid-surgery, or delay the start of the surgical procedure. In particular, the surgeon may need to halt the surgery to switch to a different operational mode of the robotic surgical system to obtain and review the desired information before resuming the procedure. And in some cases, the robotic surgical system may only enable network connectivity during brief time windows or may not enable any network access, rendering some patient information or imagery completely inaccessible during the surgical procedure. These and other limitations exist because robotic surgical systems are provided with a fixed set of functionality. If a surgeon or medical center wishes to enhance the default functionality of the robotic surgical system, they must contact the vendor and either pay an upgrade fee to enhance the functionality, sponsor development of new functionality, or simply proceed without the enhancement if it is not available.

To address these shortcomings, robotic surgical systems according to this disclosure allow enhanced functionality, referred to as “apps,” to be added from third-parties, e.g., vendors other than the supplier of the robotic surgical system. These apps may be installed by the end user and then activated during a surgical procedure to provide additional functionality to the surgeon. However, because these third-party apps are not provided by the equipment vendor, there are potential compatibility issues as well as risks attendant with third-party software, such as malware, security holes, bugs, undesirable yet intended behavior, exfiltration of sensitive information, etc. In addition to these risks apps may be overly-intrusive onto available screen space, include pop-up information, contain distracting audible or visual notifications, or otherwise impede the safe performance of the surgical procedure.

To allow robotic surgical systems to be enhanced with third-party apps, while addressing these security, safety, and usability concerns, an example robotic surgical system includes a discrete computing device that may be connected to one or more surgical robots. The computing device receives a new third-party app, which includes a cryptographically signed deployment manifest. The computing device reads the deployment manifest to verify the cryptographic signature in the manifest to ensure the app is authentic and to identify resources that are required to execute the app. Once the app is verified, the computing device can create a restricted execution environment, e.g., a sandbox, based on the deployment manifest and the app's required resources, e.g., display requirements, audio requirements, storage requirements, network access requirements, etc.

Access to these resources is then provided by one or more ports exposed by the sandbox and made available to the app according to the configurations set out in the manifest. The app may then be made available for use on the surgical robot's user interface, e.g., by providing a selectable icon to activate the app. When the app is launched, the corresponding sandbox environment may be initiated and the app executed within it. The sandbox may then provide the specified resources to the app, via one or more ports, subject to restrictions applied to those ports, such as limited access to external network communication, chair position, generator activation, ergonomic settings, power state control (e.g., shutdown, restart, power on, etc.), app launching, endoscopic illumination, video multiplexing, etc., managing what can be displayed and when, e.g., pop-up notifications, what sounds are available, how often notifications may be provided, etc. These limitations may be applied to the exposed ports themselves or by another process, e.g., a display manager for the robotic surgical system, that receives video data from the respective port and manages its presentation on a display screen. Because these resources are pre-established in the manifest file, and because the sandbox manages access to the resources, the app is restricted from accessing unauthorized resources. Further, because the app is executed within its own execution environment, should the app crash or otherwise terminate unexpectedly, it will not impact the other functionality within the robotic surgical system.

Systems and methods according to this disclosure enable the use of functionality on a robotic surgical system that has been developed by third parties other than the vendor of the robotic surgical system. These enhancements are enabled while mitigating or preventing risks associated with such flexibility, including security risks (e.g., unauthorized access or exfiltration of patient data, denial of service attacks), safety risks (e.g., interrupting or interfering with robotic surgical instruments, distracting the surgeon), and usability risks (e.g., data loss, slow or inefficient processing, cluttering on-screen real estate or saturating the user with notifications). By enabling the robotic surgical system to employ one or more restricted execution environments to install and run third-party apps through the use of signed manifests and customized execution environments, the functionality of the robotic surgical system can be increased while minimizing the risks mentioned above.

This illustrative example is given to introduce the reader to the general subject matter discussed herein and the disclosure is not limited to this example. The following sections describe various additional non-limiting examples and examples of robotic surgical systems enabling third-party application execution in a restricted environment.

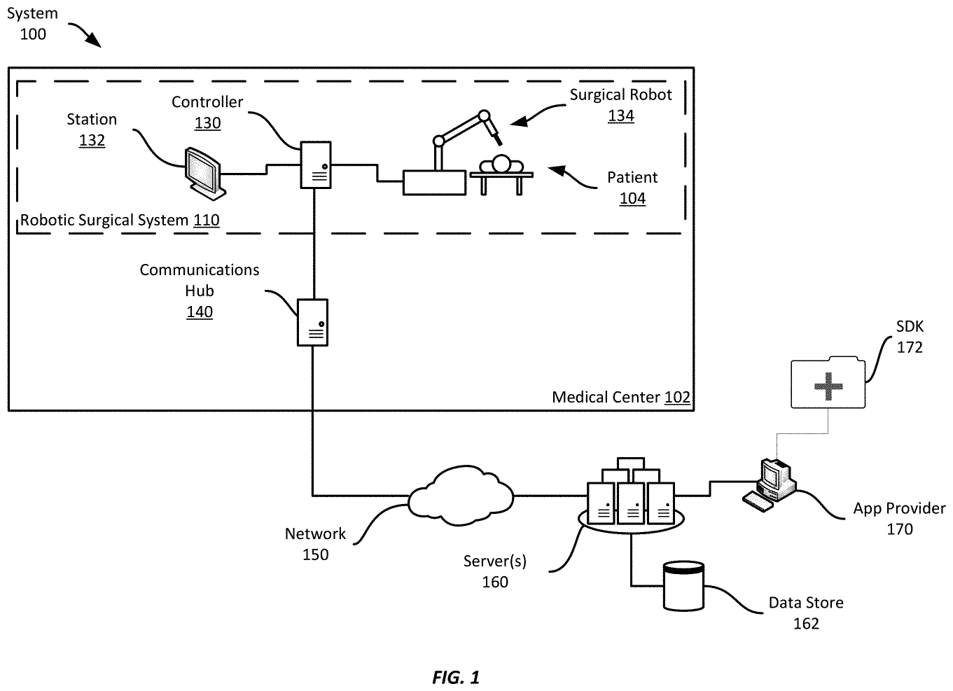

Referring now to FIG. 1, FIG. 1 shows an example surgical system 100 enabling third-party application execution on robotic surgical systems. In this example, the system 100 includes components located within a medical center 102, as well as other components located at any suitable location, including remotely from the medical center 102. Located within the medical center 102 are a communications hub 140 and a robotic surgical system 110, which includes a station 132, controller 130, and a surgical robot 134. However, it should be appreciated that the medical center 102 may have more than one robotic surgical system 110, station 132, controller 130, patient 104, or surgical robot 134. Further, the surgical robot 134 shown in medical center 102 may be communicatively coupled to portions of the robotic surgical system 110 that are remotely located from the medical center 102. For example, a virtual private network (not shown) can be used to allow for remote access to the surgical robot 134, enabling a telesurgical procedure.

Outside of the medical center 102 are a communications network 150, e.g., the Internet, remote server(s) 160 and an associated data store 162, which may include one or more databases or other servers. An app provider 170 is a software vendor or developer that develops third-party applications using a software development kit (“SDK”) 172. Software vendors using SDK 172 can create apps that use standardized software interfaces to enable installation and use on robotic surgical systems. These apps can then be sent to remote server(s) 160 and are available to download and install onto the robotic surgical system 110 via network 150 and communications hub 140. And while the network 150 is depicted as being external to the medical center 102 in this example, in some examples, part or all of the network may be located within the medical center 102.

As discussed above, the robotic surgical system 110 includes a controller 130, a surgical robot 134, and a station 132 usable by personnel in the OR, such as to view surgery information, video, apps, etc., from the surgical robot 134, which may be used to operate on a patient 104 (though the patient is not part of the robotic surgical system 110). The controller 130 is in communication with an optional communications hub 140 that enables, optimizes, or improves communication to the remote server(s) 160 via the network 150. In addition, the communications hub 140 provides access to patient or other medical information stored locally at the medical center 102. Further, in some examples the communications hub 140 may operate as a remote server, such as in one example in which the communications hub 140 is not local to the medical center 102.

The surgical robot 134 is any suitable robotic system that can be used to perform surgical procedures on a patient 104. A surgical robot 134 may have one or more articulating arms connected to a base. The arms may be manipulated by a controller 130, which may include one or more user interface devices, such as joysticks, knobs, handles, or other rotatable or translatable devices to effect movement of one or more of the articulating arms. The articulating arms may be equipped with one or more surgical instruments to perform aspects of a surgical procedure. Different surgical robots 134 may be configured for particular types of surgeries, such as cardiovascular surgeries, gastrointestinal surgeries, gynecological surgeries, transplant surgeries, neurosurgeries, musculoskeletal surgeries, etc., while some may have multiple different uses. As a result, different types of surgical robots, including those without articulating arms, such as for endoscopy procedures, may be employed according to different examples.

In some examples, surgical robots (or a respective controller, e.g., controller 130) may be configured to record, share, or access data during a surgical procedure. For example, the surgical robot 134 may record inputs made by the user, actions taken by the surgical robot 134, times (e.g., timestamps) associated with each input or action, video from one or more cameras of the surgical robot, etc. In some examples, the surgical robot 134 may include one or more sensors that can provide sensor signals, such as thermocouples, pulse sensors, SvO2 or SpO2 sensors, one or more cameras, etc., or other information to be recorded, such as temperatures, pulse information, images, video, etc. Such information may be obtained by the sensor and transmitted to a computing device within the surgical robot 134 itself or to the controller 130 for storage. Furthermore, while only one surgical robot 134 is depicted, any suitable number of surgical robots may be employed within a robotic surgical system 110.

The controller 130, in this example, includes a computing device in communication with the surgical robot 134 and is able to control access and use of the robot. For example, the controller 130 may require that a user authenticate herself before allowing access to or control of the surgical robot 134. As mentioned above, the controller 130 may include, or have connected to it, one or more user input devices capable of providing input to the controller 130, such as a keyboard, mouse, or touchscreen, capable of controlling the surgical robot 134, such as one or more joysticks, knobs, handles, dials, pedals, etc. In some examples, the functionality of controller 130 and station 132 can be combined into a single computing device.

As mentioned above, the app provider 170 develops third-party apps using SDK 172. Software vendors such as app provider 170 can obtain the SDK 172, e.g., a devkit, from a trusted entity (not shown), to create portable and verifiable apps that can be cryptographically signed by the trusted entity to prove the authenticity of the app. In addition to providing a check on authenticity, the trusted entity's signature can also provide an assurance of quality for the application, e.g., by verifying the application operates as expected within a surgical robotic environment and does not interfere with performance during a surgery. Thus, a cryptographic signature may be withheld by a trusted entity, e.g., a provider of a surgical robotic system, even if an app is developed using a valid version of the SDK. The SDK 172 provides a framework with which to build apps specific to the platform of the robotic surgical system 110, and may include one or more toolkits, libraries, guidelines, application programming interfaces (“APIs”), etc. to enable developers to create apps that are whitelisted, e.g., conform with the requirements specific to the software platform of the robotic surgical system 110. In some examples, the SDK 172 employed by an app provider may provide one or more SDK libraries to be included with the software app when distributed.

In some examples, SDK 172 can include a framework, one or more libraries, forms designers, resource editors, etc., which are used to provide functionality to the app, such as access to file systems or display devices, user interfaces, etc. For example, SDK 172 may include an SDK library that translates an app's request, e.g., an API call to end a sound can be translated into a network request to the appropriate IP address and port to the service providing access to the targeted resources. In this example, the end sound call will be executed if and only if the app has been provided with a port connected to an audio output device to provide end sound functionality. Otherwise, the end sound call will fail, and in some examples, a socket command, e.g., a command to open or use a network connection will return a failure message to the SDK 172 library. Further, in some examples, the SDK library may then interpret such a failure of the socket open connection and return a failure message to the app that made the API call.

Alternatively, or in addition to, the SDK 172 framework can employ one or more proxy services (not shown) to provide apps with their requested resources via a single provided port, rather than providing different ports for different requested resources. For example, instead of using SDK libraries to provide fine-grain control for each app's request, as discussed above, the SDK can enable communications with a proxy service to execute remote procedure calls (RPC) from an app. The RPCs are used by the app to request access to particular functionality. In some examples, such a proxy service can perform a verification of the permissions for a requesting app for each RPC request. The proxy service can then issue a separate RPC to perform the requested action upon verification, or if the app is not allowed to employ the requested functionality, the proxy service can return a failure message.

Further, in some examples, the computing capabilities of the proxy service can be integrated within one computing device for the entire system, one computing device for each app, or any other suitable configuration. In one example, the proxy service can remove the need to determine an app's permissions during a runtime environment setup, enabling the SDK library to set up communications with the proxy service when the app is launched. And in another example, the proxy service may check an app's permissions for every RPC request, instead of once, e.g., during the initial setup of the execution environment.

As discussed above, the SDK library has the expanded functionality of providing end sound call functions. However, the SDK library can provide other additional external functionality, such as sending display instructions, a notification, audio, the ability to read data. In some examples, an SDK library can also provide additional functions to be performed outside of the execution environment of an app. In one such example, the SDK library may have the ability to access certain protected storage.

The SDK 172 enables app provider 170 to write software apps to be executed on the robotic surgical system 110. As the developer creates the app, she may access resources, e.g., storage, a display device, an audio device, etc., that would require permission from the execution environment. Thus, the SDK may track the usage of such resources to enable creation of a manifest file that specifies the resources needed from the sandbox. When the developer builds the app for testing or deployment, the app provider 170, using the SDK 172, can then also generate a corresponding manifest file to cause the app's sandbox to create and provide corresponding ports to provide access to the needed resources. However, in some examples, the SDK or a development environment may automatically generate a manifest based on resources employed by the app.

Once an app is created using the SDK 172, an app provider 170 can have the app and the manifest file cryptographically signed by a trusted entity. The trusted entity uses private keys to sign the app and manifest file to allow the app and manifest file to be verified by the end user as authentic and unaltered. The app's installation package may include an executable file, resource files, and a manifest file such as the deployment manifest discussed above. The combination or bundle of files contained within the app's installation package are all signed together with a single private key from trusted entity. In addition to, or alternatively, an SDK library included in the app bundle may be cryptographically signed with the app or sent separately, retaining its cryptographic signature from the trusted entity's original SDK 172. Further, the app and the manifest file may be separately signed rather than sharing a single signature for the entire installation package.

A cryptographically signed app, created using the SDK 172, can be downloaded and installed on a robotic surgical system 110. In some examples, the cryptographic signature of the app bundle is verified upon downloading or installation of the app. And, in some examples, the cryptographic signature of the app bundle is verified before launching the app every time, thereby preventing an app provider 170 or a malicious actor from modifying the app itself or the app's manifest file. Such modifications would result in the cryptographic signature no longer matching the installed app or corresponding manifest file. In other examples, an app that malfunctions can have its cryptographic signature revoked, e.g., by revoking a corresponding certificate. For example, the controller 130 may verify the app's signature in part by determining whether a corresponding certificate is present on a certificate revocation list.

After verification of an app's signature during installation, a request to launch the app requires a similar verification of the app for each launch request, verifying its signature as discussed above. For example, the controller 130 creates a restricted execution environment for an app based on requested resources present in the app's manifest. In some examples, based on the app's manifest, the restricted execution environment may only allow the app to access its own local file storage. In other examples, the restricted execution environment may limit the app's usage of memory, restrict the initiation of network connections, block API calls, or implement any other system constraint. The app's ability to access resources, e.g., to initiate network connections of the controller 130, are based on an approved list of requested ports in the manifest. The robotic surgical system 110 uses the app's approved resources as parameters to configure and instantiate the restricted execution environment, e.g., by running a set up script, prior to running the app. In some examples, the restricted execution environment can be created in a sandbox in a processing device such as the controller 130, in a separate processing device such as station 132, or in a remotely located processing device, e.g., in the communications hub 140 or the remote server(s) 160.

In some examples, the controller 130 may use the app's manifest to determine an app's requested resources. The controller 130 identifies the resources requested by an app to configure a restricted execution environment for the app based on the resources identified in the manifest file. For example, the app's manifest may indicate a request to access a networked resource, a display device, an audio device, non-volatile storage, etc., or any combination of these. The controller 130 can then determine whether the app may or may not access the requested resources. In the ordinary course, if the app and manifest are both authenticated via their signatures, the app will be provided with the specified resources by way of one or more ports provided in the execution environment. However, in some scenarios, certain resources may be disabled on a robotic surgical system, e.g., according to an administrator's configuration. For example, surgeon's may complain about audio notifications from apps, and so the app's access to such resources may be disabled, despite the resources being specified in the manifest file.

Further, the controller 130 may use the app's manifest to determine an app's requested privileges. Some app providers 170 may include requested privileges in the app's manifest. For example, app providers 170 may request privileges to receive data from sensors, calendars, cameras, location, usage, microphones associated with the surgical robot 134. The controller 130 reads the verified manifest to determine whether those privileges should be granted, e.g., based on the configuration of the surgical robotic system.

As discussed above, a surgeon using the surgical robot 134 can access an app using the robotic surgical system 110. An authenticated user such as the surgeon may access a software app via the controller 130, launching the app in the restricted execution environment. The third-party app can provide additional functions to enhance the abilities of the surgical robot 134. For example, an app may overlay graphics onto an image of an anatomical part, allow access to electronic health records (EHR), enable three-dimensional virtualization, present multi-angled views of an image, allow telecommunication with remotely-located surgeons, etc. The app, running in the restricted execution environment, can provide such functionality using approved resources listed in its manifest.

In some examples, the controller 130 employs an app's manifest to create a restricted execution environment for the app. For example, the controller 130 may use an access level specified in the verified manifest requested to determine the users that can access the app via the surgical robot 134. In some examples, the manifest's identified access level may indicate that only medical personnel can access the app. In other examples, the app's manifest may restrict access to a group of medical personnel, e.g., only surgeons, a single surgeon, the chief surgeon, a hospital administrator, etc. In still further examples, the app may require a user to have specialized privileges to access the app.

It should be appreciated that while this example shows only one surgical robot 134 and controller 130 in communication with the communications hub 140, in some examples, the communications hub 140 may be in communication with multiple controllers 130 and surgical robots 134. For example, the medical center 102 may have one communications hub 140 per floor, or one for every four surgical robot 134/controller 130 combinations, etc. In some examples, the medical center 102 may only have a single communications hub 140 that is in communication with all controllers 130 and surgical robots 134 at the medical center 102.

The robotic surgical system 110 is in communication with one or more remote servers 160 via network 150. The network 150 may be any combination of local area networks (“LAN”), wide area networks (“WAN”), e.g., the Internet, etc. that enable electronic communications between the communications hub 140 and the remote servers 160.

The remote server(s) 160, in conjunction with the data store 162, store third-party apps. In some examples, the remote server(s) 160 may act as app server(s), providing apps to the robotic surgical system 110, as well as updates to software previously transmitted to the robotic surgical system 110, connectivity to other registered robotic surgical systems 110, remote software functionality, etc.

In some examples, the remote server(s) 160 can also function similarly to communications hub 140, storing information specific to one or more medical centers 102. Such information may include a list of approved third-party applications, permission or access levels associated with medical personnel, frequently requested information by approved third-party apps, records about one or more surgeries to be performed, previously performed surgeries, medical personnel, medical centers, operating rooms, patients, etc., to enable a user to request third-party apps, create new surgeries, schedule them, assign medical personnel, assign a patient 104, allocate an OR and a robotic surgical system 110 for the surgery, etc. Thus, the remote server(s) 160 can provide management and administrative control over the access to third-party apps, the access to data related to surgeries, and the access to data during those surgeries.

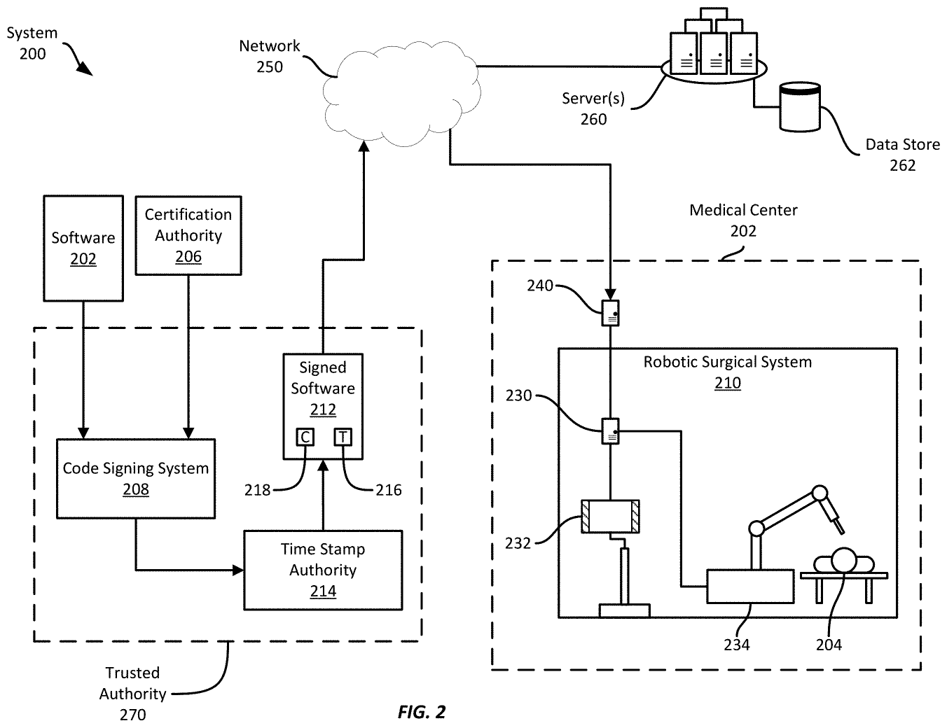

Referring now to FIG. 2, FIG. 2 shows an example surgical system 200 enabling third-party application execution on robotic surgical systems. In this example, the system 200 includes medical center 202 having components of a robotic surgical system 210 communicatively coupled to and located remotely from components of a trusted authority 270. Similar to the medical center 102, medical center 202 is communicatively coupled to remote server(s) 260 and an associated data store 262 via a network 250, which are substantially similar to remote server(s) 160, data store 162, and network 150, respectively and may include one or more databases or other servers. Additionally, the robotic surgical system 210, like the robotic surgical system 110, includes a controller 230, station 232, and surgical robot 234 for operating on a patient 204. The robotic surgical system 210 communicates with communications hub 240, which may include a remote server, to access network 250. As discussed above, the surgical robot 234 shown may communicatively couple portions of the robotic surgical system 210 that are remotely located.

The code signing system 208 of the trusted authority 270 receives software 202, e.g., SDKs, APIs, or apps, and a digital certificate that certifies ownership of a cryptographic key from a certification authority 206. The code signing system 208 verifies the authenticity of the certificate, validates the software, and signs the digital certificate before optionally providing the signed certificate 218 to a time stamp authority 214. The time stamp authority 214 can issue a trusted timestamp 216, certifying the authenticity of the signed digital certificate associated with the software. The signed software 212, having the signed digital certificate 218 and optional timestamp 216 can then be transmitted by the trusted authority 270, or another trusted entity, to the remote server(s) 260, data store 262, or communications hub 240 via network 250.

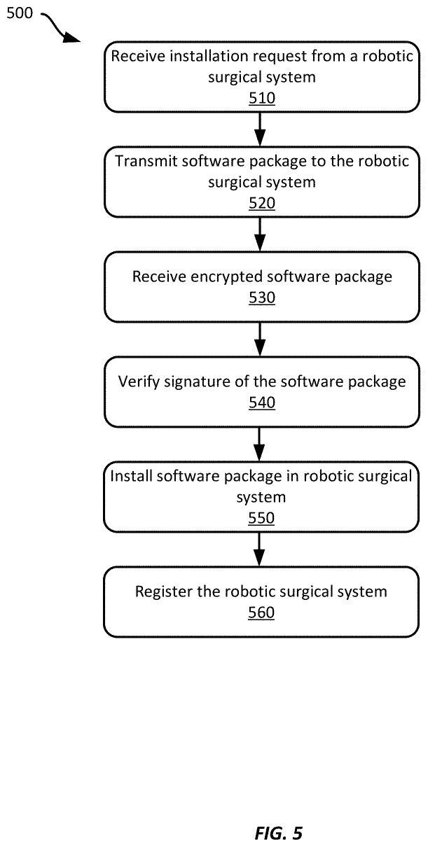

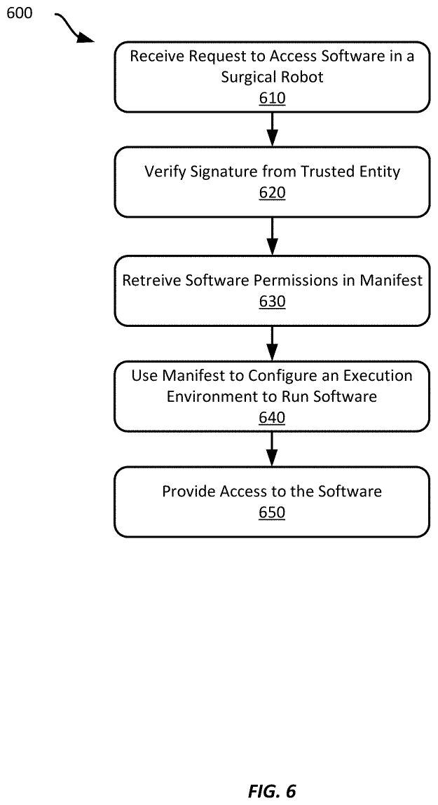

As will be discussed in detail below, with respect to the example methods in FIGS. 5 and 6, the robotic surgical system 210 receives the signed software. Controller 230 can then verify the authenticity of signed software 212, using the signed digital certificate 218 and optional timestamp 216, before installing the signed software 212 in the robotic surgical system 210. Once installed, the software 212 can be launched in an execution environment by the controller 230 using a sandbox, jail, virtual execution environment, e.g., a virtual machine, or any other suitable isolated execution environment.

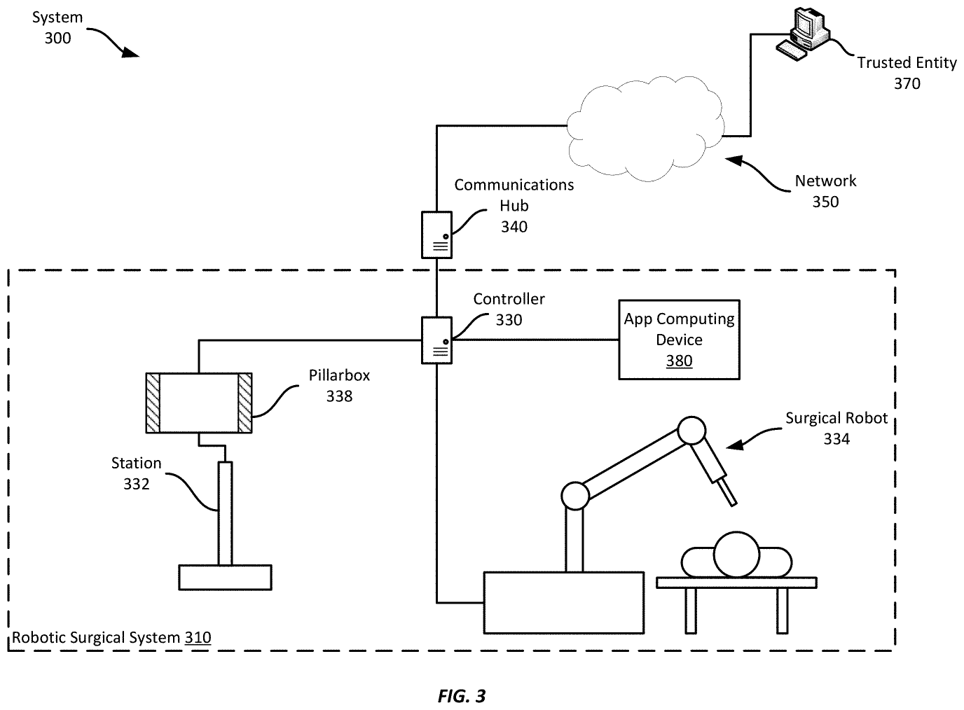

Referring now to FIG. 3, FIG. 3 shows an example surgical system 300 enabling third-party application execution on robotic surgical systems. In this example, the system 300 includes a trusted entity 370 connected to network 350 and communications hub 340. The trusted entity 370 may provide important information to the communications hub 340, including encryption keys, certificates of authenticity, timestamps, verification information for requested apps, etc. As discussed above, communications hub 340 can access, compile, and send vital hospital records, personnel and patient information, surgical scheduling information, as well as third-party apps to the controller 330 of a robotic surgical system 310. The robotic surgical system 310 includes controller 330, which communicates with station 332, the surgical robot 334 and app computing device 380 to display surgical information to the medical personnel in the OR.

In this example, app computing device 380 is a separate computing device from controller 330 that stores and executes apps in restricted execution environments using a separate processor or processors (not shown). The execution environments provided by app computing device 380 enables apps to be executed in an environment that ensures the separate, mission critical processing performed by the controller 330, station 332, and surgical robot 334 are not degraded, decelerated, or otherwise inhibited or interrupted. In some examples, the app computing device 380 can be used to perform verification, validation, or testing of new software apps, updates, or other developmental software code.

The diagonal lines shown in the display of the station 332 are pillarboxes 338, which are vertical portions of the widescreen display. In some examples, the pillarboxes 338 may only be present on the display of station 332 during a surgical procedure. In other examples, the pillarboxes 338 may provide a space for third-party apps. It should be appreciated that the vertical pillarboxes 338 are just one arrangement of the display of the station 332. In some examples, only one side of the display may have a pillarbox 338, the pillarboxes 338 may be replaced by one or more horizontal letterboxes, four-directional windowboxes, transparent or semi-transparent overlays, graphics or graphical overlays, etc.

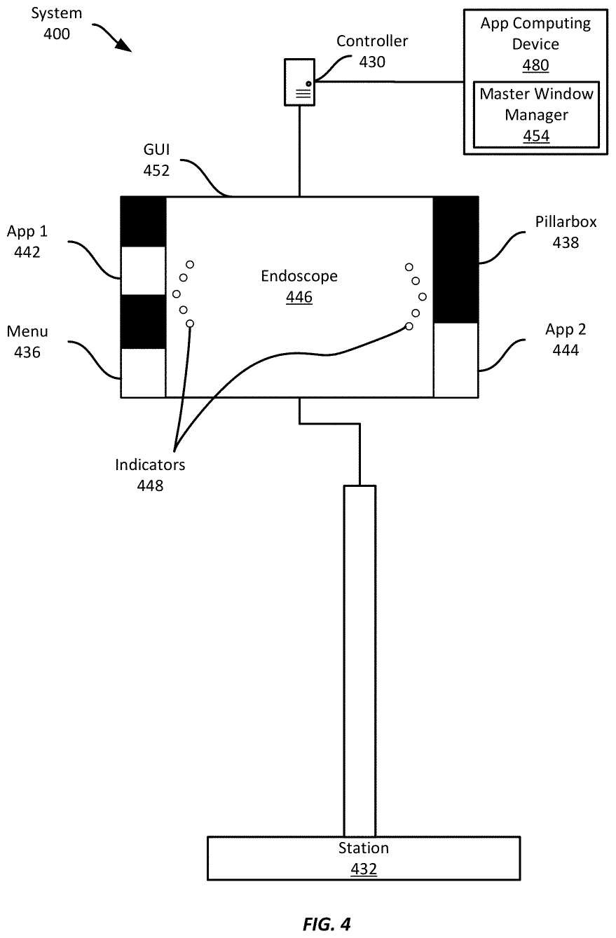

Referring now to FIG. 4, FIG. 4 shows an example system 400 of a robotic surgical system enabling third-party application execution on robotic surgical systems. The system 400 includes a controller 430 and an app computing device 480, which are communicatively coupled to station 432. Controller 430 is substantially similar to controller 330, discussed above with respect to FIG. 3, however, in this example, app computing device 480 includes a master window manager 454 and station 432 shows a display screen showing a graphical user interface (“GUI”) 452 having multiple subparts. The center portion 446 of the display provides video from an endoscope. Other subparts of the GUI 452 provide output from other sources, such as App 1 442 and App 2 444. The pillarbox 438 represents display space that is available but unused. The GUI 452 also includes on-screen indicators 448, which are overlays on the GUI 452 and enable a user to access various, selectable functionality. In some examples, the station 432 may include a controller (e.g., controller 330), one or more additional computing devices (not shown), a tablet, a laptop, a mouse, a keyboard, or any other suitable input device.

The master window manager 454 can ensure apps' access to screen real estate is strictly controlled and cannot be overridden. By determining a corresponding display location on the screen for a particular source, and scaling and displaying the received stream within the corresponding display location, the master window manager 454 can prevent apps from obfuscating, or otherwise interfering with, the endoscopic view 446. For example, the master window manager 454 can control the size and location of a video output region for App 1 442, and App 2 444. The master window manager 454 may also restrict or control other functionality, such as audio output or interactivity, e.g., via a touch screen. And while the master window manager 454 is depicted as being executed on the same computing device as apps, in some examples, the master window manager 454 may be executed on a separate computing device is communicatively connected to the app computing device 480.

The endoscope view 446 represents the surgeon's view of an endoscopic camera, which can provide a live feed of an endoscopy, a near real-time feed, a previously recorded surgical procedure, a surgical simulation in a virtual or augmented reality environment, etc. The endoscope view 446 can display images captured by any communicatively coupled endoscope camera. During surgery, the endoscope 446 provides mission critical information to the surgeon that typically cannot be safely interrupted, especially while a surgeon manipulates a surgical robot, e.g., making an incision. To ensure interruptions do not occur, the station 432 or controller 430 may include a master window manager, such as master window manager 454, that determines what to display and where based on configuration settings.

As discussed above, the master window manager 454 controls the layout and arrangement of information displayed on the display screen. The master window manager 454 may determine the arrangement of App 1 442 and App 2 444 based on predetermined or default settings, which may be implemented at the direction of one or more users. For example, a robotic surgical system may designate a specific level of permissions to a user, e.g., a chief surgeon may have a higher level of permissions than another physician or other medical personnel that enables the chief surgeon to establish an arrangement of displayed information on the display of the station 432.

The menu 436 can provide access to the master window manager 454 and to launch various apps such as App 1 442 and App 2 444. A user with a sufficient level of access can change the location and size of App 1 442 and App 2 444, and by extension the location and size of pillarbox 438. Alternatively, or in addition to, the menu 436 or other input devices can provide controls to employ the master window manager 454 to exit an apps view, enabling the endoscope 446 to occupy the full screen display of station 432.

Exemplary apps, App 1 442 and App 2 444, operate within the confines of their respective execution environments. When running simultaneously, App 1 442 and App 2 444 are executed in separate computing environments. However, if only one of App 1 442 or App 2 444 is being executed by the robotic surgical system, the computing device that creates the restricted execution environment need not create a second execution environment. Instead, if the computing device determines the port requirements are the same for both App 1 442 and App 2 444, then the computing device can simply re-use that existing restricted execution environment for the app being launched for either App 1 442 or App 2 444. But if the computing device determines the port requirements are different for App 1 442 and App 2 444, then the computing device may create or use different execution environments, or the computing device may modify an existing restricted execution environment to conform with the requested app's port requirements.

Similar to the pillarboxes 338 shown in FIG. 3, the black pillarboxes 438 shown in FIG. 4 are vertical portions of the widescreen display of the station 432. In some examples, the master window manager 454 controls the pillarboxes 438, which may provide a predetermined amount of spacing between apps outside of the endoscope 446. In some examples, an authorized user can use the master window manager 454 to alter the content of pillarboxes 438, e.g., by reducing or increasing its size, eliminating them completely by increasing the sizes of App 1 442 and App 2 444, replace them with additional apps, or maximize their size. In some examples, the size and location of the pillarboxes 438, like the menu 436, App 1 442 and App 2 444, and endoscope 446, may be predetermined or fixed according to procedures or policies of the medical center. It should be appreciated that the vertical pillarboxes 438, and the menu 436, App 1 442, and App 2 444 that overlay portions of vertical pillarboxes 438 are just one arrangement of the display of the station 432.

Referring now to FIG. 5, FIG. 5 illustrates an example method 500 for a robotic surgical system enabling third-party application execution in a restricted environment. The example method 500 will be discussed with respect to the system 100 shown in FIG. 1, however, it should be appreciated that example methods according to this disclosure may be employed with any suitable system according to this disclosure.

At block 510, remote server(s) 160 receive an installation request from the robotic surgical system 110 for software. In this example, the robotic surgical system 110 is new to a medical center 102 and is requesting installation of a third-p arty app on the surgical robot 134; however, it should be appreciated that installing a third-party app may be performed at any suitable time. As a result, the robotic surgical system 110 sends the installation request to the remote server(s) 160 to obtain one or more available software packages.

At block 520, the remote server(s) 160 transmits the requested one or more signed software installation packages to the robotic surgical system 110. As discussed above, the remote server(s) 160 and associated data store 162 may receive and retain signed apps from one or more app providers 170.

At block 530, the controller 130 of the robotic surgical system 110 receives an encrypted software package from the remote server(s) 160. The encrypted software package can include a software app, a manifest, and additional information, such as help files, user's manuals, etc. If multiple installation requests for software were issued, the controller 130 receives a software package in response to each request. However, in some examples, a software package may include multiple software apps and corresponding manifests. Further, in some examples, the software package may not be encrypted.

At block 540, the controller 130 verifies the signature of the encrypted software package. In this example, the controller 130 employs public key cryptography (“PKC”) to perform the decryption and ensure the validity of the digital signature of the software package received. In some examples, the controller 130 may perform the PKC decryption technique on a hash of data, checksum, or any other string of data associated with the software package instead of the entire software package. In other examples, the controller 130 can decrypt the software package based on any suitable encryption technique, such as an asymmetric key algorithm, e.g., digital signature standard (“DSS”), Rivest-Shamir-Adleman (“RSA”), YAK, ElGamal, Diffie-Hellman (“DH”), etc.

Alternatively, or in addition to, the decryption and verification of the cryptographic signature of the software package may be performed by the communication hub 140 or the remote server(s) 160 before reaching the controller 130 of the robotic surgical system 110. In some examples, e.g., the dedicated app computing device 380 shown in FIG. 3, can perform the decryption and verification of the software package. Further, in some examples, the computing capabilities of the controller 130 can be integrated within a station, such as the station 432 discussed above with respect to FIG. 4. Such a station can have sufficient processing capabilities to perform the decryption and verification of the digital signature.

At block 550, the controller 130 installs the software package in the robotic surgical system 110, such as by extracting one or more files from an archive or executing an installation script. In addition, the controller 130 identifies a manifest file of the software package that identifies the resources required by the installed app, which is described below in greater detail at block 630 of FIG. 6.

At block 560, the controller 130 registers the robotic surgical system 110 including the installed software package with the remote server(s) 160. Once the software package is successfully installed by the controller 130, the controller 130 may provide a notification of the installation to the remote server(s) 160.

Referring now to FIG. 6, FIG. 6 illustrates an example method 600 for enabling third-party application execution on robotic surgical systems. The example method 600 will be discussed with respect to the system 100 shown in FIG. 1, however, it should be appreciated that example methods according to this disclosure may be employed with any suitable system according to this disclosure.

At block 610, the controller 130 receives a request to access a third-party software app installed on the surgical robot 134. In this example, the surgical robot 134 has at least one third-party app installed, as described above with respect to FIG. 5.

At block 620, the controller 130 verifies the signature of the requested software in the robotic surgical system 110. After receiving the request to access the software app, the controller 130 may optionally verify the digital signature of the signed software again, including the manifest file, using any technique discussed with respect to block 540 illustrated in the example method 500 of FIG. 5. Alternatively, the controller 130 may determine verification of the app is not required based on a previous verification or recorded usage data associated with the app. In some examples, the controller 130 verifies the signature of the software package, including the signed manifest, has not been revoked by comparing the signature to a revocation list.

In some examples, the controller 130 can verify the trusted entity information by validating a trusted digital signature of the trusted entity information. Similar to block 540 of the example method 500 illustrated in FIG. 5, the controller 130 verifies the trusted digital signature of the manifest. In this example, the controller 130 employs a second PKC, DSS, RSA, YAK, ElGamal, DH, asymmetric key algorithm, or any other suitable cryptographic technique, to perform the decryption and verification of the manifest. In some examples, the controller 130 may perform the validation technique using a hash, checksum, or any other string or datum associated with the software package.

At block 630, the controller 130 retrieves the software permissions requested in the manifest. As discussed above, with respect to block 550 of the example method 500 illustrated in FIG. 5, after the controller 130 verifies the authenticity of the digital signature of the software package, the controller 130 reads the contents of the software package to ensure the contents of the software package conform to the platform of the robotic surgical system 110. Specifically, the controller 130 identifies a manifest file of the software package that includes an app's name, version, license, trusted entity information, requested resources, requested privileges, security level, dependencies, etc. The controller 130 then verifies the version of the software package is capable of being run on the version of the environment available on the robotic surgical system 110.

Alternatively, or in addition to, the decryption and verification of the cryptographic signature of the manifest may be performed by the communication hub 140 or the remote server(s) 160 before reaching the controller 130 of the robotic surgical system 110. In some examples, e.g., the dedicated app computing device 380 shown in FIG. 3, can decrypt and verify the cryptographic signature of the manifest. Further, in some examples, the computing capabilities of the controller 130 can be integrated within a station, such as the station 432 discussed above with respect to FIG. 4. Such a station can have sufficient processing capabilities to perform the decryption and verification of the digital signature.

At block 640, the controller 130 of the robotic surgical system 110 uses the signed manifest to configure an execution environment to run the software. As mentioned above, the app's manifest provides identifying information such as the app's name, version, license, trusted entity information, etc. This information is important to verify the provenance and authenticity of the app and its contents. However, the app's manifest also includes the app's requested resources, requested privileges, security level, and any other relevant information, which is used to create an execution environment for the app to run successfully. For example, the manifest may specify certain resources needed by the app, such as a display area, audio output capability, data storage, access to user input devices, etc.

In some examples, an app's manifest may indicate that a user must have permission, e.g., a requisite access level, before the app can be launched. In such an example, accessing the app may require authentication of a user's access level by a username and password, personal identification number, key card, voice identification, facial recognition, fingerprint scanning, implantation device, challenge response, a combination of these, or any other suitable single-factor or multi-factor authentication technique. At a high level, the controller 130 may restrict specific users from accessing an app based on the user's access level.

As discussed above, with respect to FIG. 1, the controller 130 may use the app's manifest to determine an app's requested privileges in response to requests from app providers 170. The controller 130 reads the verified manifest and may determine whether the app requires such privileges to function properly and whether those privileges should be granted. An SDK library requesting such privileges from the controller 130 can identify the app's permission level, allowing the controller 130 to determine whether the privilege should be granted.

It should be appreciated that the examples discussed with respect to block 630 may be employed in any suitable combination discussed herein.

At block 650, the controller 130 provides access to the software app. After the controller 130 verifies the signed software in the manifest, the controller 130 provides access to the software app in an execution environment determined above. In this example, the controller 130 may change the limitations placed on the restricted execution environment based on the determinations discussed above, increasing or decreasing restrictions on the execution environment. In some examples, the controller 130 may establish the restricted execution environment by creating an execution environment on a remote computing device and executing the app within that execution environment.

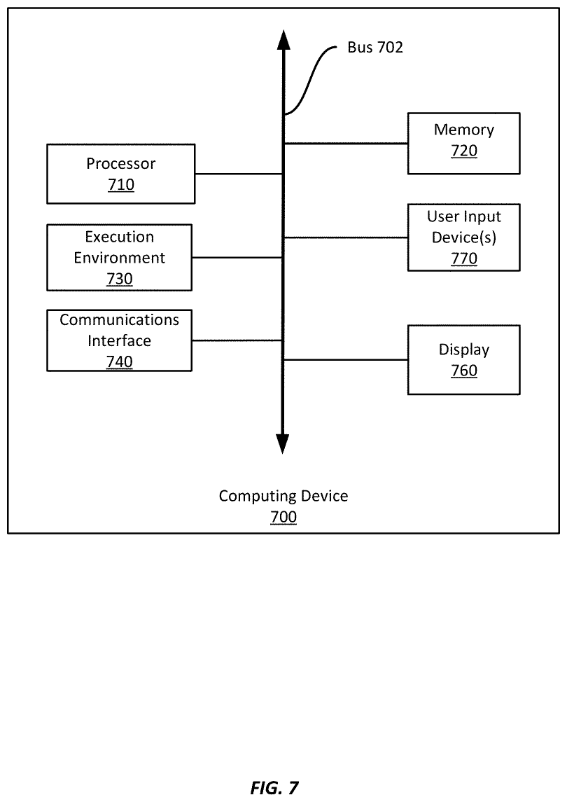

Referring now to FIG. 7, FIG. 7 shows an example computing device 700 for a robotic surgical system enabling third-party application execution in a restricted environment. The computing device 700 includes a processor 710 in communication with other hardware via a bus 702. A memory 720, which can be any suitable tangible (and non-transitory) computer-readable medium such as random access memory (“RAM”), read-only memory (“ROM”), erasable and electronically programmable read-only memory (“EEPROMs”), or the like, embodies program components that configure operation of the computing device 700. In the embodiment shown, computing device 700 further includes one or more user input devices 770, an execution environment 730, a communications interface 740 and a display 760.

It should be appreciated that computing device 700 may also include additional processors, additional storage, and a computer-readable medium (not shown). The processor(s) 710 may execute additional computer-executable program instructions stored in memory. Such processors may include a microprocessor, digital signal processor, application-specific integrated circuit, field programmable gate arrays, programmable interrupt controllers, programmable logic devices, programmable read-only memories, electronically programmable read-only memories, or other similar devices.

The computing device 700 includes an execution environment 730 that enables the controller 130 to creates a restricted execution environment for an app. As discussed above, the creation of the execution environment 730 may be based on requested resources present in the app's manifest. In some examples, the execution environment 730 may only allow the app to access its own local file storage, may limit the app's usage of memory, initiate network connections, make API calls, or implement another system constraint. In some examples, the execution environment 730 is a restricted execution environment, e.g., a sandbox in the controller 130, a separate processing device, or a remotely located processing device such as the communications hub 140 or the remote server(s) 160.

In some examples, the execution environment 730 employs the resources requested by an app to restrict the execution environment 730 based on resources identified in the manifest file. The execution environment 730 may restrict audio notifications from apps, disabling such requested resources, despite the resources being specified in the manifest file. In addition to, or alternatively, the execution environment 730 can enable access to an app's requested resources generally as discussed above, e.g., app providing or enabling receipt of sensor data, calendar information, camera data, location data, usage data, or audio data associated with one or more microphones of the surgical robot 134. And as discussed above, when the surgical robot 134 accesses an app using the robotic surgical system 110, the app is launched in the execution environment 730.

The computing device 700 also includes a communications interface 740. In some examples, the communications interface 740 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

The methods, devices, and systems discussed above are examples. Various configurations may omit, substitute, or add various procedures or components. For example, in alternative configurations, the methods may be performed in a different order. In another example, the methods may be performed with fewer steps, more steps, or in combination. In addition, certain configurations may be combined in various configurations. As technology evolves, many of the elements are examples and do not limit the scope of the disclosure or claims.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

That which is claimed is:

1. A computing device, comprising:
a processor; and
a non-transitory computer readable medium comprising processor-executable instructions to cause the processor to:
receive a request to execute an application from a robotic surgical system;
obtain a cryptographic signature associated with a manifest, the manifest associated with the application;
in response to verification of the manifest or the application based on the cryptographic signature, determine at least one configuration setting for the application based on the manifest, the at least one configuration setting comprising a permission;
configure an execution environment based on the at least one configuration setting, the at least one configuration setting enabling the application to interact with the robotic surgical system; and
execute the application in the execution environment.

2. The computing device of claim 1, further comprising processor-executable instructions to cause the computing device to:
restrict the execution environment of the application by limiting communication between the robotic surgical system and the computing device, wherein the computing device comprises a separate processor, a distributed computing environment, or a separate execution environment in a separate device.

3. The computing device of claim 1, further comprising processor-executable instructions to cause the computing device to:
authenticate a user;
determine an access level for the user based on one or more user permissions;
verify the user is allowed access based on the access level; and
launch the application when the one or more user permissions are verified.

4. The computing device of claim 1, further comprising processor-executable instructions to cause the computing device to:
verify the cryptographic signature with a first trusted entity; and
install the application on the computing device, wherein the installation only occurs after verification that the cryptographic signature originated from the first trusted entity or a second trusted entity.

5. The computing device of claim 1, wherein the cryptographic signature comprises an encryption key, wherein the encryption key is provided by a first trusted entity, and further comprising processor-executable instructions to cause the computing device to:
verify the cryptographic signature using the encryption key from the first trusted entity; and
install the application on the computing device, wherein the installation only occurs after verification that the cryptographic signature originated from the first trusted entity or a second trusted entity and is unaltered.

6. The computing device of claim 1, further comprising processor-executable instructions to cause the computing device to:
receive a request to access a resource via one or more external ports; and
allow the application to access the resource based on the execution environment associated with the robotic surgical system via the one or more external ports.

7. The computing device of claim 1, further comprising processor-executable instructions to cause the computing device to:
identify one or more external ports requested by the application;
determine permissions for each requested external port based on the manifest, before running the application; and
create a restricted execution environment to execute the application based on the determined permissions of the one or more identified external ports.

8. The computing device of claim 7, further comprising processor-executable instructions to cause the computing device to:
enable the application to use an existing restricted execution environment based on the determined permissions of the one or more identified external ports; and
communicate display or audio information from the application to a display manager of the robotic surgical system through the one or more external ports, the display manager controlling the display or audio information output by the robotic surgical system, and wherein the display or audio information comprises streaming audio, streaming video, annotated streaming video, an image, or an annotated image.

9. A method, comprising:
receiving, by a computing device, a request to execute an application from a robotic surgical system;
obtaining, by the computing device, a cryptographic signature associated with a manifest, the manifest associated with the application;
determining, by the computing device and in response to verification of the manifest or the application based on the cryptographic signature, at least one configuration setting for the application based on the manifest, the at least one configuration setting comprising a permission;
configuring, by the computing device, an execution environment based on the at least one configuration setting, the at least one configuration setting enabling the application to interact with the robotic surgical system; and
executing the application in the execution environment.

10. The method of claim 9, further comprising:
restricting the execution environment of the application by limiting communication between the robotic surgical system and the computing device, wherein the computing device comprises a separate processor, a distributed computing environment, or a separate execution environment in a separate device.

11. The method of claim 9, further comprising:
authenticating a user;
determining an access level for the user based on one or more user permissions;
verifying the user is allowed access based on the access level; and
launching the application when the one or more user permissions are verified.

12. The method of claim 9, further comprising:
verifying the cryptographic signature with a first trusted entity; and
installing the application on the computing device, wherein the installation only occurs after verification that the cryptographic signature originated from the first trusted entity or a second trusted entity.

13. The method of claim 9, wherein the cryptographic signature comprises an encryption key, wherein the encryption key is provided by a first trusted entity, and further comprising:
verifying the cryptographic signature using the encryption key from the first trusted entity; and
installing the application on the computing device, wherein the installation only occurs after verification that the cryptographic signature originated from the first trusted entity or a second trusted entity and is unaltered.

14. The method of claim 9, further comprising:
receiving a request to access a resource via one or more external ports; and
allowing the application to access the resource based on the execution environment associated with the robotic surgical system via the one or more external ports.

15. The method of claim 9, further comprising:
identifying one or more external ports requested by the application;
determining permissions for each requested port based on the manifest, before running the application; and
creating a restricted execution environment to execute the application based on the determined permissions of the one or more identified external ports.

16. The method of claim 15, further comprising:
enabling the application to use an existing restricted execution environment based on the determined permissions of the one or more identified external ports; and
communicating display or audio information from the application to a display manager of the robotic surgical system through the one or more external ports, the display manager controlling the display or audio information output by the robotic surgical system, and wherein the display or audio information comprises streaming audio, streaming video, annotated streaming video, an image, or an annotated image.

17. A non-transitory computer readable medium comprising processor-executable instructions to cause a processor to:
receive a request to execute an application from a robotic surgical system;
obtain a cryptographic signature associated with a manifest, the manifest associated with the application;
in response to verification of the manifest or the application based on the cryptographic signature, determine at least one configuration setting for the application based on the manifest, the at least one configuration setting comprising a permission;
configure an execution environment based on the at least one configuration setting, the at least one configuration setting enabling the application to interact with the robotic surgical system; and
execute the application in the execution environment.

18. The non-transitory computer readable medium of claim 17, further comprising processor-executable instructions to cause the processor to:
restrict the execution environment of the application by limiting communication between the robotic surgical system and a computing device, wherein the computing device comprises a separate processor, a distributed computing environment, or a separate execution environment in a separate device.

19. The non-transitory computer readable medium of claim 17, further comprising processor-executable instructions to cause the processor to:
authenticate a user;
determine an access level for the user based on one or more user permissions;
verify the user is allowed access based on the access level; and
launch the application when the one or more user permissions are verified.

20. The non-transitory computer readable medium of claim 17, further comprising processor-executable instructions to cause the processor to:
verify the cryptographic signature with a first trusted entity; and
install the application on a computing device, wherein the installation only occurs after verification that the cryptographic signature originated from the first trusted entity or a second trusted entity.

21. The non-transitory computer readable medium of claim 17, wherein the cryptographic signature comprises an encryption key, wherein the encryption key is provided by a first trusted entity, and further comprising processor-executable instructions to cause the processor to:
verify the cryptographic signature using the encryption key from the first trusted entity; and
install the application on a computing device, wherein the installation only occurs after verification that the cryptographic signature originated from the first trusted entity or a second trusted entity and is unaltered.

22. The non-transitory computer readable medium of claim 17, further comprising processor-executable instructions to cause the processor to:
identify one or more external ports requested by the application;
determine permissions for each requested port based on the manifest, before running the application; and
create a restricted execution environment to execute the application based on the determined permissions of the one or more identified external ports.

23. The non-transitory computer readable medium of claim 22, further comprising processor-executable instructions to cause the processor to:
enable the application to use an existing restricted execution environment based on the determined permissions of the one or more identified external ports; and
communicate display or audio information from the application to a display manager of the robotic surgical system through the one or more external ports, the display manager controlling the display or audio information output by the robotic surgical system, and wherein the display or audio information comprises streaming audio, streaming video, annotated streaming video, an image, or an annotated image.
